(19) United States
(12) Patent Application Publication
(10) Pub. No.: US 2026/0183486 A1
(43) Pub. Date: Jul. 2, 2026

(54) Syringe With Integrated Easy-Open Cap

(71) Applicant: Becton, Dickinson and Company, Franklin Lakes, NJ (US)

(72) Inventors: Praveen Nalawade, Bangaluru (IN); Senthilkumar Rakkiyappan, Coimbatore (IN)

(21) Appl. No.: 18/868,897

(22) PCT Filed: Jun. 15, 2023
(86) PCT No.: PCT/US23/25352

§ 371 Date: Nov. 25, 2024

(30) Foreign Application Priority Data
Jun. 21, 2022 (IN) 202211035495

(51) Int. Cl. A61M 5/31; A61M 5/14; A61M 39/10; A61M 39/20

(52) U.S. Cl. CPC A61M 5/3134; A61M 5/14; A61M 39/10; A61M 39/20; A61M 2005/1403; A61M 2039/1077

(57) ABSTRACT

A syringe includes a barrel having a proximal end, a distal end including a distal opening, and a sidewall extending between the proximal end and the distal end. The syringe further includes a plunger positioned in the barrel for expelling fluid from the barrel through the distal opening and a cap connected to the distal end of the barrel including a connector defining a flow channel through the cap. The cap is movable relative to the barrel between a first position, where fluid flow through the distal opening of the barrel to the flow channel of the connector is prevented, and a second position, where fluid flow through the distal opening of the barrel to the flow channel of the connector can occur.

### CROSS-REFERENCE TO RELATED APPLICATION

The present application claims priority to Indian Patent Application number 202211035495 entitled “Syringe with Integrated Easy-Open Cap” filed Jun. 21, 2022, the entire disclosure of which is hereby incorporated by reference in its entirety.

### BACKGROUND OF THE INVENTION

### Field of the Invention

The present disclosure relates generally to syringes, such as prefilled syringes, and, in particular, to a syringe having an integrated cap, which can be easily opened by a user, preferably using one hand.

### Description of Related Art

Conventional syringes comprise a barrel having an open proximal end and an opposed distal end. A distal tip, sometimes referred to as a luer, projects from the distal end of the syringe barrel and includes a narrow passage or channel in fluid communication with an interior of the syringe barrel. These conventional syringes are often prefilled with medical fluids, such as flush solutions or therapeutic agents, during manufacture. When prefilled with the medical fluid, the syringe barrel can be sealed to prevent contamination of the medical fluid or loss of the medical fluid prior to use. Seals also prevent practitioners from being needlessly exposed to the medical fluid contained in the syringe barrel, which can be particularly important for toxic or otherwise harmful medications.

Some prefilled syringes have stoppers or closures, often referred to as tip caps, mounted over the distal tip of the syringe barrel for sealing the syringe barrel to prevent leakage and to avoid contamination of the medical fluid contained therein. Prior art tip caps are removed from the distal tip of the syringe barrel by the practitioner shortly before using the syringe by grasping the tip cap and pulling the tip cap axially away from the syringe. When a prefilled syringe is capped with a tip cap, it is especially important that a good seal be maintained. This is usually achieved by tightly affixing the tip cap to the distal tip of the syringe barrel. However, when overly tightened, the tip cap can be difficult to remove or may be damaged. Further, a prefilled syringe may be autoclaved after filling and capping so that the prefilled syringe is contained within sterile packaging. The autoclaving procedure can cause interactions between the tip cap and the syringe barrel, which can further increase the difficulty in removing the tip cap from the distal tip of the syringe barrel.

Due to the tight connection between the tip cap and distal tip of the syringe barrel, the practitioner may be required to apply a substantial gripping force to remove the tip cap from the distal end of the syringe barrel. Tightly grasping the tip cap can be difficult for some practitioners who may lack physical strength and dexterity needed to correctly grip the tip cap, especially considering the small size of the typical tip cap. Further, when tightly grasping the tip cap, the practitioner's fingers are in close proximity to the distal tip of the syringe barrel. Accordingly, there is a risk that the practitioner will contact the distal tip of the barrel while removing the tip cap, which can contaminate the distal tip of the syringe barrel.

Once the tip cap is removed, which occurs shortly before a procedure is performed, the luer tip at one end of the syringe barrel is exposed to a non-sterile environment, which provides additional opportunities for undesirable contamination. Further, in some cases, practitioners may remove the tip cap to perform a preparatory action (e.g., to flush air from the syringe barrel) and then reattach the same tip cap to the syringe distal tip prior to performing a fluid delivery procedure. This reattachment of the tip cap is yet another opportunity for contamination of the distal tip of the syringe barrel.

Prefilled syringes are often connected to and used to deliver fluid to a patient through vascular access devices (VADs), such as intravenous (IV) catheters (e.g., peripheral catheters or central venous catheters). If not properly maintained or if exposed to non-sterile environments, which, as described previously, can occur as the practitioner manipulates or removes the tip cap from the syringe barrel, the VADs can become contaminated, sealed with blood clots, and/or can spread infection. Many medical facilities implement sterile practices and protocols to avoid contamination and to ensure that VADs are used properly and do not become sealed or infected. These protocols often include sterilizing medical devices including the VAD and flushing the catheter with a flush solution. Specifically, VAD standards of practice usually recommend flush procedures be performed after catheter placement, before fluid infusion, and before and after drug administration, blood sampling, transfusions, and/or administration of parenteral nutrition. These flush procedures are intended to confirm catheter patency, avoid drug incompatibilities, ensure that the complete drug dose is administered to the patient, prevent thrombus formation, and minimize a risk of bloodstream infections caused by contamination of the VAD.

While sterilization procedures implemented by medical facilities certainly address some contamination risks, improved designs for medical devices and tools that reduce opportunities for contamination and simplify device use are needed. In particular, syringe designs that ensure that the distal tip of the syringe barrel remains sterile and free from contamination throughout a fluid delivery procedure using the VAD will address many current contamination problems that arise during an infusion procedure. Further, syringe and cap designs that are easy to open or remove and which can be manipulated without touching the syringe distal tip will reduce common contamination risks for conventional prefilled syringes. The syringes and integrated caps design of the present disclosure addresses these issues.

### SUMMARY OF THE INVENTION

According to an aspect of the disclosure, a syringe includes a barrel having a proximal end, a distal end including a distal opening, and a sidewall extending between the proximal end and the distal end. The syringe further includes a plunger positioned in the barrel for expelling fluid from the barrel through the distal opening; and a cap connected to the distal end of the barrel with a connector defining a flow channel through the cap. The cap is movable relative to the barrel between a first position, where fluid flow through the distal opening of the barrel to the flow channel of the connector is prevented, and a second position, where fluid flow through the distal opening of the barrel to the flow channel of the connector can occur.

According to another aspect of the disclosure, a prefilled flushing syringe includes: the previously described syringe; and a predetermined volume of a fluid disposed in the barrel of the pre-filled syringe. The cap of the previously described syringe is initially in the first position, thereby preventing the fluid in the barrel from passing through the open distal end portion of the barrel to the flow channel of the connector.

According to another aspect of the disclosure, a method for expulsion of fluid from the previously described syringe includes: moving the cap of the previously described syringe from the first position to the second position, such that fluid flow from the barrel to the flow channel of the connector is permitted; attaching the connector of the cap to a vascular access device; and moving the plunger through the barrel to expel the fluid from the barrel and through the flow channel of the connector to the vascular access device.

Non-limiting illustrative examples of embodiments of the present disclosure will now be described in the following numbered clauses.

Clause 1: A syringe comprising: a barrel comprising a proximal end, a distal end comprising a distal opening, and a sidewall extending between the proximal end and the distal end; a plunger positioned in the barrel for expelling fluid from the barrel through the distal opening; and a cap connected to the distal end of the barrel comprising a connector defining a flow channel through the cap, wherein the cap is movable relative to the barrel between a first position, where fluid flow through the distal opening of the barrel to the flow channel of the connector is prevented, and a second position where fluid flow through the distal opening of the barrel to the flow channel of the connector can occur.

Clause 2: The syringe of clause 1, wherein the cap rotates relative to the barrel to move from the first position to the second position.

Clause 3: The syringe of clause 1 or clause 2, wherein the cap rotates about a longitudinal axis of the barrel to move from the first position to the second position.

Clause 4: The syringe of any of clauses 1-3, wherein the cap is configured to be manually moved from the first position to the second position.

Clause 5: The syringe of clause 4, wherein the syringe is capable of single-handed operation, both to move the cap from the first position to the second position and to expel fluid from the barrel through the flow channel of the connector by moving the plunger through the barrel.

Clause 6: The syringe of any of clauses 1-5, wherein the barrel comprises at least one of polyester, polycarbonate, polypropylene, polyethylene, polyethylene terephthalate, or acrylonitrile butadiene styrene.

Clause 7: The syringe of any of clauses 1-6, wherein the barrel further comprises an annular flange about the proximal end of the barrel for grasping the barrel and plunger to move the plunger through the barrel.

Clause 8: The syringe of any of clauses 1-7, wherein the plunger comprises a stopper positioned in the barrel and a plunger rod extending proximally from the stopper through the proximal end portion of the barrel.

Clause 9: The syringe of clause 8, wherein the stopper comprises a thermoplastic elastomer, such as isoprene.

Clause 10: The syringe of clause 8 or clause 9, wherein the stopper comprises a proximal end, a distal end, and an outer surface extending between the proximal end and the distal end that seals against an inner surface of the barrel.

Clause 11: The syringe of any of clauses 8-10, wherein the plunger rod comprises a connector portion inserted into and engaged to a cavity of the stopper for securing the stopper to the plunger rod.

Clause 12: The syringe of any of clauses 1-11, wherein the connector of the cap comprises at least one of a luer connector, a threaded connector, or a snap connector.

Clause 13: The syringe of any of clauses 1-12, wherein the connector is tapered, such that an outer diameter of a proximal end of the connector is wider than an outer diameter of a distal tip of the connector.

Clause 14: The syringe of any of clauses 1-13, further comprising at least one seal positioned between the distal opening of the barrel and the cap, the at least one seal comprising at least one hole for fluid flow from the barrel to the flow channel of the connector.

Clause 15: The syringe of clause 14, wherein the at least one hole of the at least one seal is offset from a longitudinal axis of the syringe and is not aligned with the distal opening of the barrel.

Clause 16: The syringe of clause 14 or clause 15, wherein the at least one hole of the at least one seal is smaller than the distal opening of the barrel.

Clause 17: The syringe of any of clauses 14-16, wherein the at least one seal comprises at least two holes, each of which are offset from a longitudinal axis of the syringe.

Clause 18: The syringe of any of clauses 14-17, wherein the at least one seal comprises a disk formed from a thermoplastic elastomer comprising the at least two holes, which are spaced apart from a longitudinal axis of the barrel and are not aligned with the distal opening of the barrel.

Clause 19: The syringe of clause 18, wherein the disk comprises a proximal ridge extending from a proximal surface of the disk positioned in a corresponding groove of the distal end of the barrel and a distal ridge extending from a distal surface of the disk positioned in a corresponding groove on the proximal end of the cap.

Clause 20: The syringe of any of clauses 1-19, wherein the at least one seal comprises a proximal stationary seal positioned between the distal end of the barrel and the proximal end of the cap, and a distal rotatable seal positioned within a proximal open end of the cap.

Clause 21: The syringe of clause 20, wherein the distal seal rotates along with the cap and the proximal seal remains stationary as the cap moves between the first position and the second position.

Clause 22: The syringe of clause 20 or clause 21, wherein the proximal seal and the distal seal each comprise at least one hole that is offset from the longitudinal axis of the syringe and is not aligned with the distal opening of the barrel, and wherein when the cap is in the second position, the at least one hole of the distal seal aligns with the at least one hole of the proximal seal so that fluid from the barrel passes through the holes of the proximal seal and the distal seal to the flow channel of the connector.

Clause 23: The system of any of clauses 1-22, wherein the cap further comprises a shield extending about the connector, the shield comprising an inner surface comprising threads configured to engage threads of a female connector for securing the syringe and cap to the female connector.

Clause 24: The syringe of clause 23, wherein a distal end of the connector protrudes beyond a distal end of the shield.

Clause 25: The syringe of clause 23 or clause 24, wherein the barrel further comprises an extension portion extending distally from the distal end of the barrel, and wherein the cap is connected to and at least partially enclosed by the extension portion.

Clause 26: The syringe of clause 25, wherein the extension portion comprises a proximal end extending from the distal end of the barrel, an open distal end, and an annular sidewall extending between the proximal end and the distal end.

Clause 27: The syringe of clause 26, wherein the cap comprises a locking ring configured to engage the cap so that the cap remains seated at the proximal end of the extension portion.

Clause 28: The syringe of any of clauses 25-27, wherein the sidewall of the barrel comprises a tapered portion between the distal end of the barrel and other portions of the barrel for guiding fluid in the barrel towards the distal opening of the barrel.

Clause 29: The syringe of any of clauses 25-28, wherein the cap comprises at least one tab extending radially outward from the shield for rotating the cap relative to the barrel.

Clause 30: The syringe of clause 29, wherein the extension portion comprises at least one slot, and wherein the at least one tab of the cap extends through the slot so that the at least one tab can be grasped by a user to rotate the cap relative to the barrel.

Clause 31: The syringe of clause 30, wherein the at least one slot extending from a distal end of the extension portion comprises an axial portion, for inserting the cap into the extension portion, and a circumferential portion, for rotating the cap relative to the barrel between the first position and the second position.

Clause 32: The syringe of any of clauses 25-31, wherein the extension portion further comprises protrusions on an inner surface of the extension portion for restricting rotation of a seal positioned over the distal opening of the barrel.

Clause 33: The syringe of clause 32, wherein the protrusions comprise axially extending ridges, ribs, or columns on the inner surface of the extension portion.

Clause 34: The syringe of any of clauses 1-33, wherein a direction of rotation for connecting the syringe to a female connector is opposite a direction of rotation to move the cap from the first position to the second position.

Clause 35: A prefilled flushing syringe, comprising: the syringe of any of clauses 1-34; and a predetermined volume of a fluid disposed in the barrel of the pre-filled syringe, wherein the cap is initially in the first position, thereby preventing the fluid in the barrel from passing through the open distal end portion of the barrel to the flow channel of the connector.

Clause 36: The prefilled syringe of clause 35, wherein the fluid comprises a saline flush solution.

Clause 37: The prefilled syringe of clause 35 or clause 36, wherein the fluid comprises a therapeutic agent.

Clause 38: The prefilled syringe of any of clauses 35-37, wherein the barrel contains from about 1 mL to about 50 mL of the fluid.

Clause 39: A method for expulsion of fluid from the syringe of any of clauses 1-34, the method comprising: moving the cap from the first position to the second position, such that fluid flow from the barrel to the flow channel of the connector is permitted; attaching the connector of the cap to a vascular access device (VAD); and moving the plunger through the barrel to expel the fluid from the barrel and through the flow channel of the connector to the vascular access device.

Clause 40: The method of clause 39, wherein the fluid contained in the barrel of the syringe comprises a saline flush solution.

Clause 41: The method of clause 39 or clause 40, wherein the fluid contained in the barrel of the syringe comprises a therapeutic agent.

Clause 42: The method of clause 40 or clause 41, wherein the barrel contains from about 1 mL to about 50 mL of the fluid.

Clause 43: The method of any of clauses 39-42, wherein the cap is manually moved from the first position to the second position by grasping a portion of the cap and rotating the cap relative to a longitudinal axis of the syringe.

Clause 44: The method of clause 43, wherein the cap is manually moved from the first position to the second position by grasping a portion of the cap with one hand.

Clause 45: The method of any of clauses 39-44, further comprising advancing the plunger through the barrel by a small amount prior to attaching the syringe to the VAD to remove air from an interior of the barrel.

Clause 46: The method of any of clauses 39-45, wherein attaching the cap to the VAD comprises inserting a distal tip of the connector through a septum of a fluid port of the VAD and twisting the syringe, such that threads of the syringe engage corresponding threads of a female connector of the VAD.

Clause 47: The method of clause 46, wherein the cap is moved from the first position to the second position by rotating the cap relative to the barrel in a first direction, and the cap is attached to the VAD by rotating the syringe in a second direction opposite from the first direction.

Clause 48: The method of any of clauses 39-47, wherein the connector comprises a male luer connector and the cap is attached to a female luer connector of the VAD by inserting the male luer connector into the female luer connector.

### BRIEF DESCRIPTION OF THE DRAWINGS

FIG. 1A is a perspective view of a syringe comprising an integrated cap, according to an aspect of the present disclosure.

FIG. 1B is an exploded view of the syringe and cap of FIG. 1A.

FIG. 2A is a perspective view of a barrel of the syringe of FIG. 1A, according to an aspect of the present disclosure.

FIG. 2B is a perspective view of a distal end portion of the barrel of FIG. 2A.

FIG. 2C is a perspective view of a cross-section of the distal end portion of the barrel of FIG. 2A.

FIG. 3A is a perspective view of a seal of the syringe of FIG. 1A, according to an aspect of the disclosure.

FIG. 3B is a perspective view of a cross-section of the distal end portion of the syringe showing the seal seated proximate to the distal end of the barrel, according to an aspect of the disclosure.

FIG. 4A is a perspective view of the cap of FIG. 1A, according to an aspect of the present disclosure.

FIG. 4B is a perspective view of a cross-section of the cap of FIG. 4A.

FIG. 4C is a perspective view of a seal for the cap of FIG. 4A.

FIG. 5A is a perspective view of a cross-section of the distal end portion of the syringe of FIG. 1A showing the cap in a closed position, according to an aspect of the present disclosure.

FIG. 5B is a cross-sectional view of the distal end portion of the syringe of FIG. 5A.

FIG. 5C is a perspective view of a cross-section of the distal end portion of the syringe of FIG. 1A showing the cap in an open position, according to an aspect of the present disclosure.

FIG. 5D is a cross-sectional view of the distal end portion of the syringe of FIG. 5C.

FIG. 6 is a flow chart showing steps for using a syringe comprising an integrated cap for delivering a medical fluid to a patient through a vascular access device, according to an aspect of the present disclosure.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. As used herein, the term “proximal” refers to a portion or end of a device, such as a syringe or catheter, which is grasped, manipulated, or used by a practitioner or another user. The term “distal” refers to an end or portion of the device that is farthest away from the portion of the device that is grasped, manipulated, or used by the practitioner. For example, the “proximal end” of a catheter or IV line refers to the end including a fluid port that is connected to a fluid container, such as an IV bag or syringe. The “distal end” of the catheter or IV line refers to the end that is connected to the patient. However, it is to be understood that the invention may assume alternative variations and step sequences, except where expressly specified to the contrary. It is also to be understood that the specific devices and processes illustrated in the attached drawings, and described in the following specification, are simply exemplary embodiments of the invention. Hence, specific dimensions and other physical characteristics related to the embodiments disclosed herein are not to be considered as limiting.

With reference to the figures, the present disclosure is directed to syringes 10, such as prefilled syringes, configured to be used by a practitioner for delivering a medical fluid to a patient through, for example, a vascular access device (VAD). The practitioner can be, for example, a medical technician, nurse, physician assistant, physician, or other trained or untrained clinicians or medical caregivers. The medical fluid can be a flush solution, such as saline and/or a heparin lock flush solution. An example of a saline flush solution is 0.9% sodium chloride USP for injection. An example of a heparin lock flush solution is 0.9% sodium chloride with 100 USP units of heparin sodium per mL or 10 USP units of heparin sodium per mL. Other flush solutions, as are known in the art, may also be used with the syringe 10 of the present disclosure. The medical fluid can also be a medication, a total parenteral nutrient (TPN) liquid, or another therapeutic agent used for treatment of chronic or acute conditions, as are known in the art. Exemplary therapeutic agents can include, for example, drugs, chemicals, biological or biochemical substances that, when delivered in a therapeutically effective amount to the patient, achieve a desired therapeutic effect.

As previously described, the syringes 10 of the present disclosure are intended to reduce contamination risks and to ensure that the distal tip of the syringe barrel remains sterile and free from contamination prior to and while it is connected to the VAD. Further, the syringes 10 of the present disclosure are designed for easy operation and, in particular, to avoid difficulties with removing tip caps of conventional prefilled syringes. In some particularly advantageous examples, the syringes 10 of the present disclosure can be operated by a practitioner with one hand. Specifically, the practitioner is able to open and close the syringe cap with one hand by, for example, pressing, sliding, pulling or otherwise manipulating a tab of a syringe cap to open and close the cap, as described in further detail herein. By contrast, as previously described, for conventional prefilled syringes, a practitioner needs to grasp the syringe barrel with one hand and grasp the tip cap with the other hand in order to pull the tightly fitted tip cap away from the distal tip of the syringe barrel. Once the syringe cap is opened, the practitioner is able to expel the medical fluid from the syringe barrel of the syringe 10 with one hand by, for example, pushing a plunger or stopper through the syringe barrel.

In some examples, the syringes 10 of the present disclosure can be provided as prefilled syringes containing the flush solution or other medical fluid. The prefilled syringes 10 can also include caps, clips, retainers, and/or other packaging to hold the plunger rod in place and to ensure that the flush solution or another medical fluid does not leak from the prefilled syringe 10 at unexpected times, such as during transport.

Syringe with an Integrated Cap

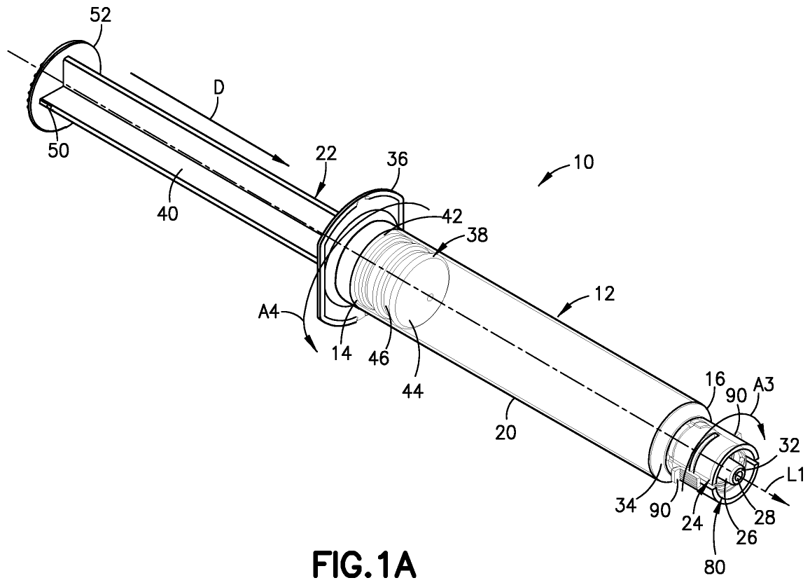

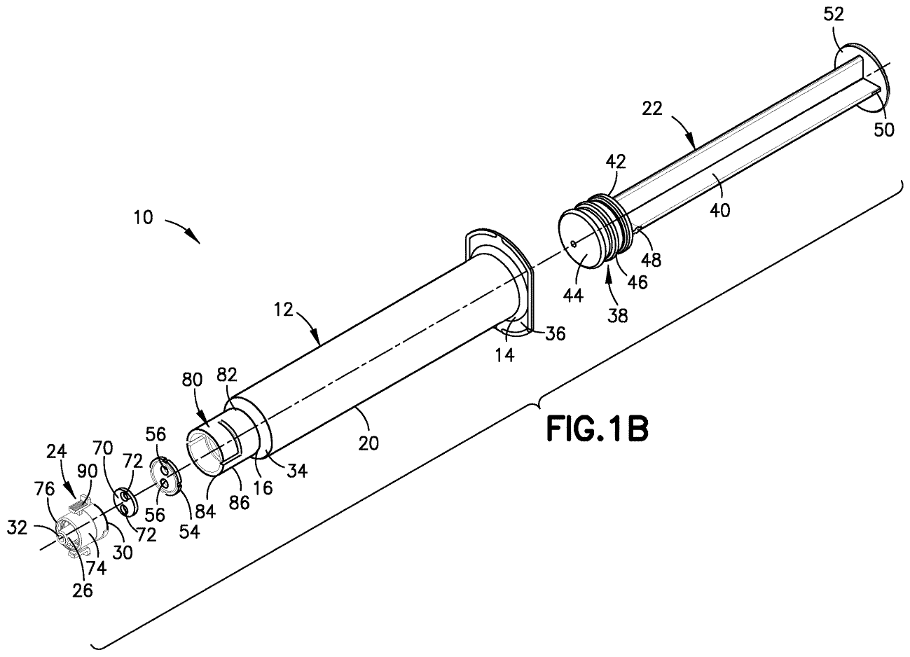

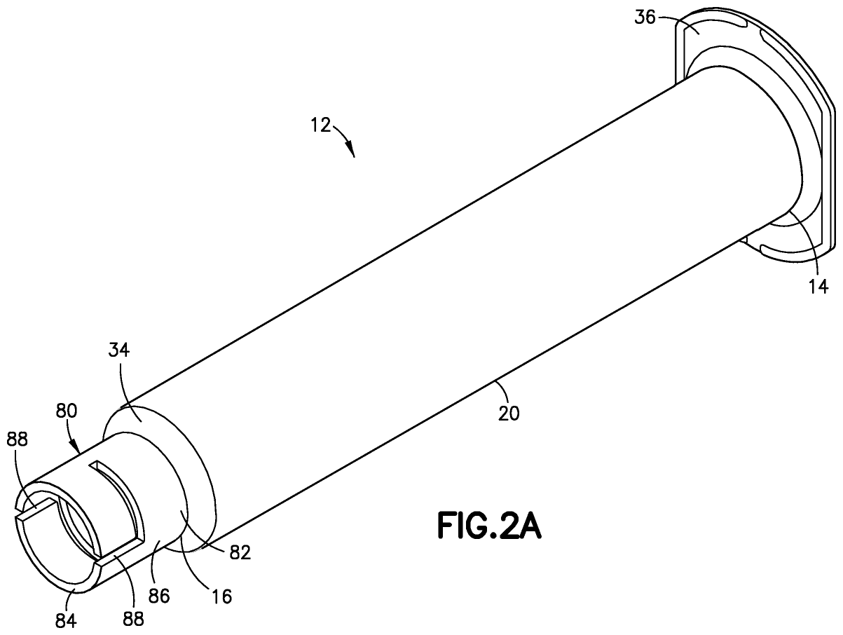

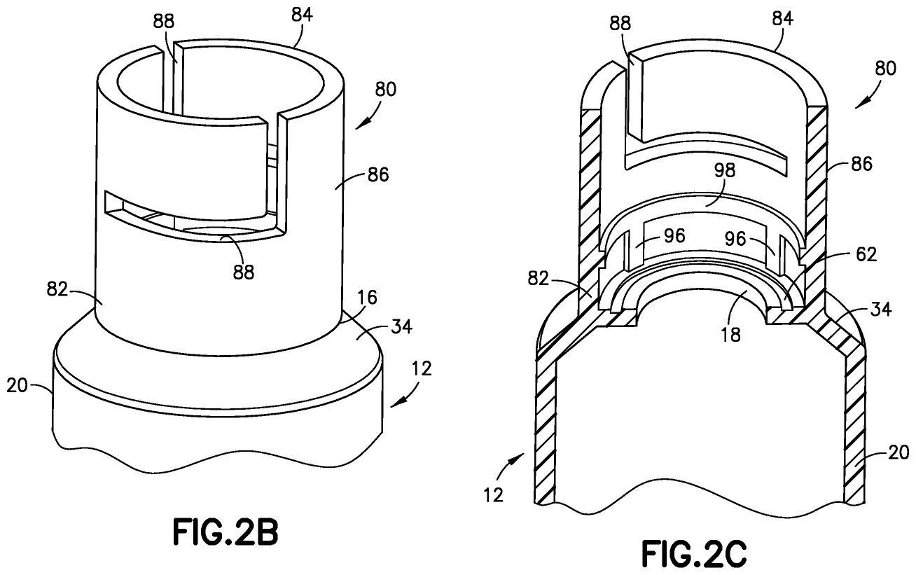

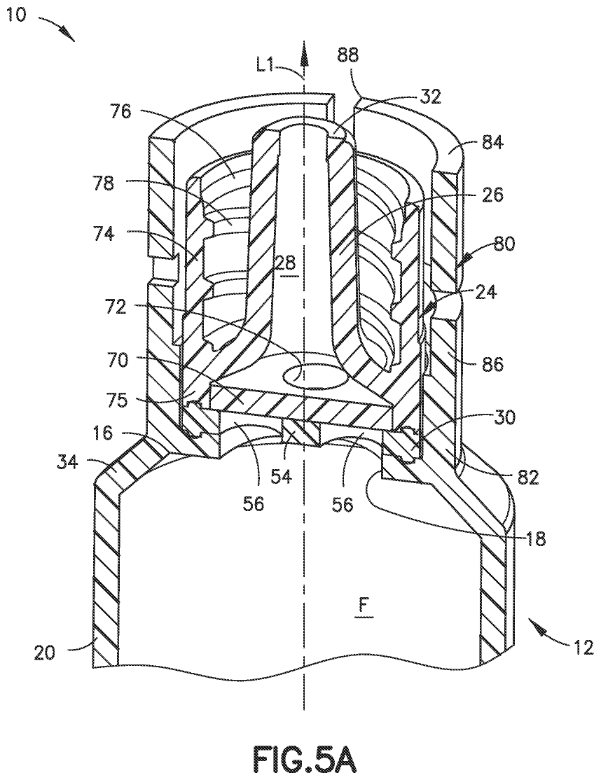

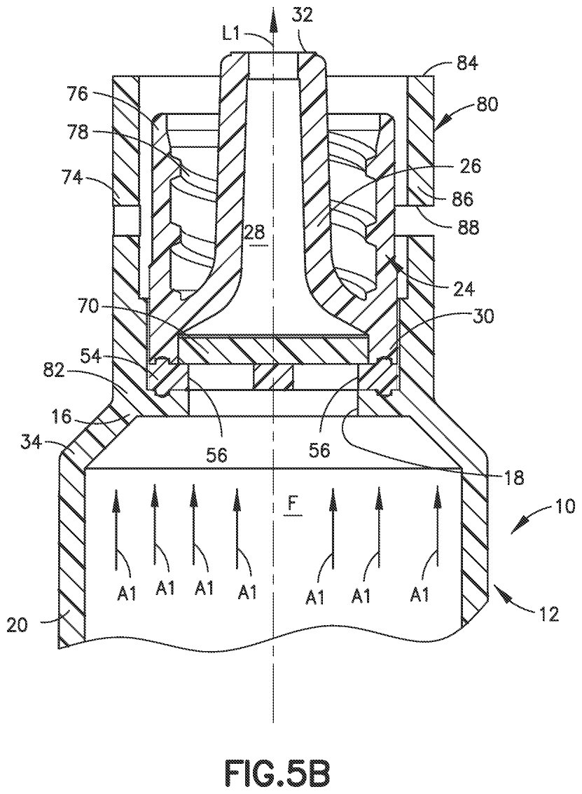

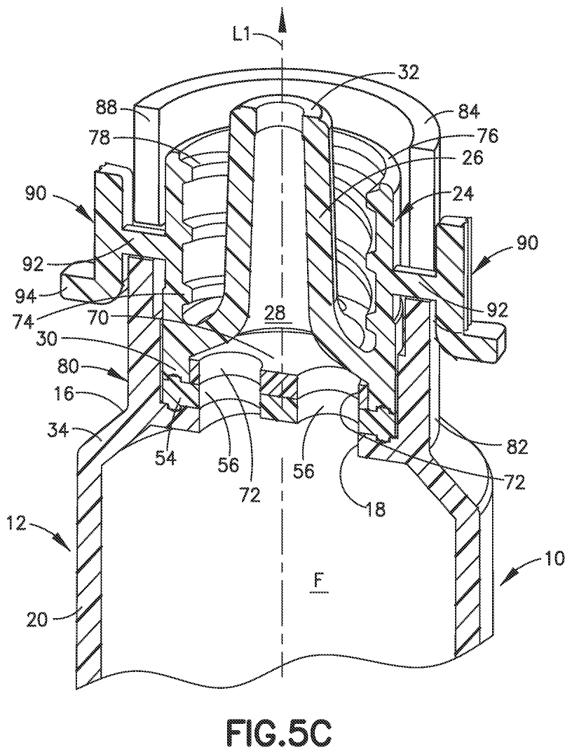

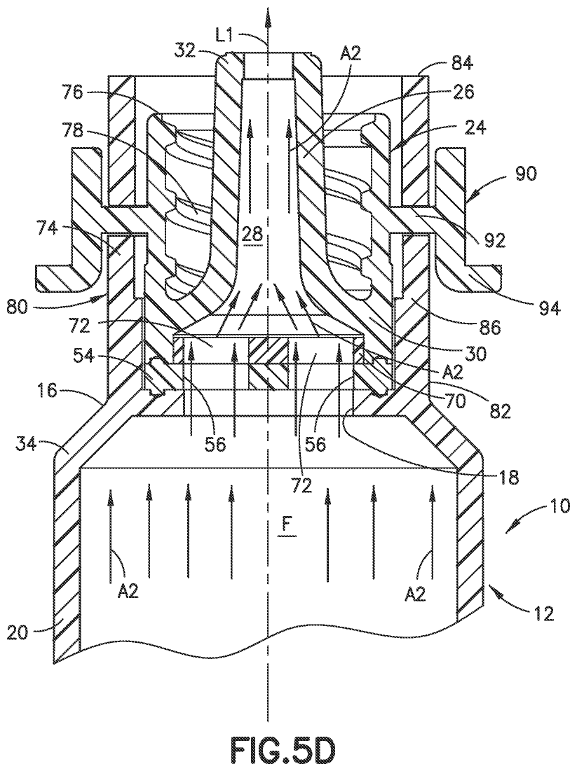

With reference to FIGS. 1A and 1B, in some examples, the syringe 10 of the present disclosure comprises a barrel 12, which can comprise a proximal end 14, a distal end 16 comprising a distal opening 18 (shown most clearly in FIG. 2C), and a sidewall 20 extending between the proximal end 14 and the distal end 16. The syringe 10 further comprises a plunger 22 positioned in the barrel 12 for expelling the medical fluid F (shown in FIGS. 5B and 5D) from the barrel 12 through the distal opening 18. The syringe 10 further comprises a cap 24, valve, cover, sealing arrangement, or similar structure connected to the distal end 16 of the barrel 12 for sealing the medical fluid F within the barrel 12 and for guiding fluid flow from an interior of the barrel 12 to expel the medical fluid F from the barrel 12. In some examples, the cap 24 comprises a connector 26 or stem extending distally from the distal end 16 of the barrel 12. The connector 26 can be an elongated and/or tubular member defining a flow passage or flow channel 28 extending from a proximal end 30 of the cap 24 to a distal end or distal tip 32 of the connector 26. As described in further detail herein, the connector 26 can be a male needleless connector 26 that is configured to be inserted into a female needless connector, such as a fluid port or opening of a VAD. For example, the cap 24 can be configured to be used with female needleless connectors, such as female luer connectors for a range of dimensions permitted by various design protocols, such as ISO 80369-7:2016 or ISO 80369-7:2021 (Female Luer Lock Connector dimensions). In some particular examples, the cap 24 can be sized to engage female needleless connectors having threads with a width at a crest of each thread of from about 0.3 mm to about 1.0 mm and a width at a root of the thread from about 0.5 mm to about 1.2 mm.

When the connector 26 is inserted into the female connector, fluid communication is established between the VAD and the interior of the barrel 12 through the cap 24. Further, the cap 24 is movable relative to the barrel 12 between a closed or first position (shown in FIGS. 5A and 5B), where fluid flow through the distal opening 18 of the barrel 12 to the flow channel 28 of the connector 26 is prevented, and an open or second position (shown in FIGS. 5C and 5D) where fluid flow through the distal opening 18 of the barrel 12 and the flow channel 28 of the connector 26 to the VAD can occur. For example, the cap 24 can be configured to twist or rotate, such as rotating about a longitudinal axis L1 (shown in FIGS. 1A and 5A-5D) of the barrel 12 and syringe 10, which moves the cap 24 from the closed position (shown in FIGS. 5A and 5B) to the open position (shown in FIGS. 5C and 5D).

In some examples, the cap 24 is configured to be manually movable, meaning that the practitioner can grasp a portion of the cap 24 and rotate, twist, slide, or otherwise move the cap 24 from the closed position to the open position. Further, as previously described, the cap 24 can be configured for single-handed operation. In particular, the practitioner desirably is able both to move the cap 24 from the closed position to the open position and to expel the medical fluid F from the barrel 12 through the flow channel 28 of the connector 26 by moving the plunger 22 through the barrel 12 using one hand.

With continued reference to FIGS. 1A and 1B, as well as FIGS. 2A-2C, the barrel 12 can be a conventional fluid-delivery syringe barrel used for medical procedures made by, for example, an injection molding process. For example, the barrel 12 can be substantially similar in shape, size, and configuration to barrels of syringes used for administering a flush solution to a VAD, as are known in the art. In some examples, the barrel 12 can be a cylindrical or elliptical prism structure formed from a rigid thermoplastic material, such as polyester, polycarbonate, polypropylene, polyethylene, polyethylene terephthalate, acrylonitrile butadiene styrene, or other injection moldable or formable resin materials, as are known in the art. Exemplary barrels 12 for flush syringes are described, for example, in U.S. Patent Appl. Pub. No. 2020/0061297, entitled “Flush Syringe Assembly with Controlled Pulsatile Flushing,” which is incorporated herein by reference in its entirety. Dimensions of the syringe barrel 12 can be determined based on the type of fluid injection being performed. In particular, the barrel 12 can be configured to contain a variety of fluid volumes depending upon the type of fluid delivery procedure being performed. For example, the barrel 12 can define an interior that contains about 1 mL to about 50 ml of the medical fluid or, preferably from about 5 mL to 20 mL of the medical fluid.

In some examples, the sidewall 20 of the barrel 12 comprises a tapered portion 34 (shown in FIGS. 2B and 2C) proximate to the distal end 16 of the barrel 12. For example, the tapered portion 34 can be positioned between the distal end 16 of the barrel 12 and other portions of the sidewall 20 of the barrel 12. The tapered portion 34 can be configured to guide fluid in the barrel 12 towards the distal opening 18 of the barrel 12 for expelling the medical fluid in the barrel 12 through the distal opening 18 towards the flow passage or flow channel 28 of the connector 26. The tapered portion 34 can be shaped to reduce dead space having a sloped or angled surface corresponding with a distal surface of a stopper or plunger 22, which moves through the interior of the barrel 12, to ensure that the fluid moves through the barrel 12 to the distal opening 18. Reducing or eliminating dead space between the stopper or plunger 22 and the distal end 16 of the barrel 12 helps to ensure that the appropriate or expected amount of fluid is expelled from the barrel 12. In some examples, the barrel 12 further comprises a finger flange 36 or grip. For example, as shown in FIGS. 1A and 1B, the finger flange 36 can extend about the proximal end 14 of the barrel 12 for grasping the barrel 12 and plunger 22 to move the plunger 22 through the barrel 12.

With continued reference to FIGS. 1A and 1B, the syringe 10 further comprises the plunger 22 configured to move through an interior of the syringe barrel 12 for aspirating a medical fluid into the interior of the syringe barrel 12 (if the syringe 10 is not a prefilled syringe 10) and/or for expelling the fluid F from the barrel 12 through the flow passage or flow channel 28 of the connector 26. The plunger 22 can comprise a stopper 38 and a plunger rod 40 connected to and extending from the stopper 38 and through the proximal end 14 of the barrel 12. The stopper 38 can have many features of conventional syringe stoppers or plungers, as are known in the art. For example, the stopper 38 can comprise a thermoplastic elastomer material, such as polypropylene or polyethylene, as well as from synthetic or natural rubber (e.g., isoprene). Further, the stopper 38 can comprise a proximal surface 42 or proximal end, a distal surface 44 or end, and an outer peripheral surface 46 extending between the proximal surface 42 and the distal surface 44. The outer peripheral surface 46 can be configured to seal against an inner surface of the sidewall 20 of the barrel 12 for moving the medical fluid F through the barrel 12. In some examples, the distal end or distal surface 44 of the stopper 38 can be tapered having a slope or angle that matches the tapered portion 34 of the sidewall 20 of the barrel 12, as previously described.

The plunger 22 also includes the plunger rod 40 connected to the stopper 38. The plunger rod 40 can be, for example, an injection molded part formed from a rigid thermoplastic material, such as polyester, polycarbonate, polypropylene, polyethylene, polyethylene terephthalate, or another thermoplastic material, as are known in the art. The plunger rod 40 can include a distal end 48 engaged to the stopper 38. For example, the distal end 48 of the plunger rod 40 can include a connector (not shown) that is inserted into a corresponding cavity or slot on the proximal surface 42 of the stopper 38. The plunger rod 40 can also include a proximal end 50 protruding proximally through the proximal end 14 of the barrel 12 and a body extending between the proximal end 50 and the distal end 48 of the plunger rod 40. The proximal end 50 of the plunger rod 40 can include a thumb press plate 52 for manipulating the plunger rod 40 to move the plunger rod 40 and stopper 38 through the syringe barrel 12. The body of the plunger rod 40 can have a variety of cross-sectional shapes and configurations within the scope of the present disclosure. For example, the body can have a generally cross shaped cross-section. In other examples, the cross-section of the plunger rod 40 can be an I-beam shape, hollow circle, hollow square, hollow rectangle, or L-shaped.

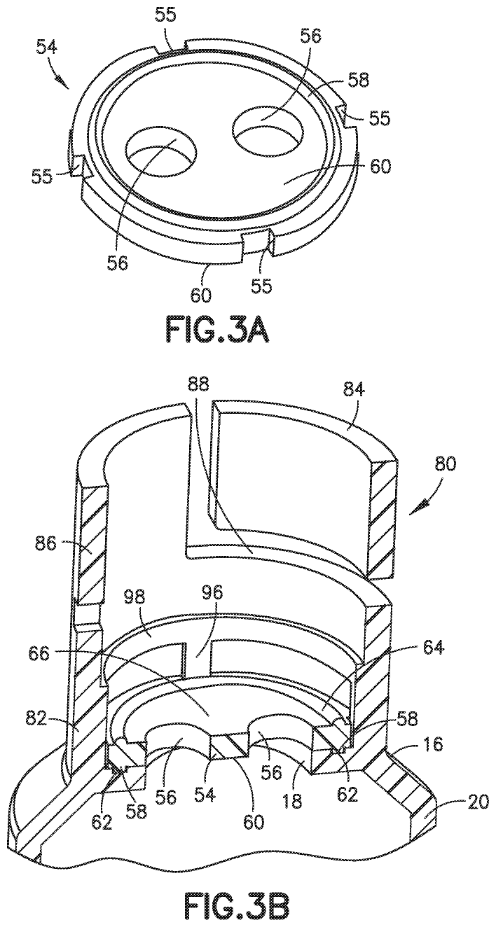

In some examples, the syringe 10 further comprises a sealing portion or seal (referred to herein as a first or proximal seal 54 and shown most clearly in FIG. 3A) for sealing the distal opening 18 of the barrel 12 to ensure that fluid passing through the distal opening 18 of the barrel 12 passes to the flow passage or flow channel 28 of the connector 26. The proximal seal 54 can be positioned between the distal opening 18 of the barrel 12 and the cap 24. In some examples, the proximal seal 54 comprises a substantially flat sheet or disk of a fluid-impermeable material, such as silicone, rubber, isoprene, or a similar flexible elastomer that seals the distal end 16 and distal opening 18 of the barrel 12. The proximal seal 54 can comprise one or more through holes 56, openings, or fluid ports for fluid flow from the barrel 12 to the flow channel 28 of the connector 26. For example, as shown in FIG. 3A, the proximal seal 54 includes two holes 56. The holes 56 of the proximal seal 54 can be offset from a longitudinal axis L1 (shown in FIGS. 1A and 5A-5D) of the syringe 10 and may not be aligned with the distal opening 18 of the barrel 12, meaning that the distal opening 18 of the barrel 12 and the holes 56 of the proximal seal 54 are not centered on a same axis. Further, as shown, for example, in FIG. 3A, the holes 56 of the proximal seal 54 can be smaller (e.g., can have a smaller diameter and/or area) than the distal opening 18 of the barrel 12. Due to the arrangement of the distal opening 18 of the barrel 12 and holes 56, fluid flow from the barrel 12, through the holes 56, to the flow channel 28 follows a somewhat curved path, as shown by arrows A2 in FIGS. 5C and 5D.

In some examples, the proximal seal 54 includes structures for maintaining position of the proximal seal 54 over the distal opening 18 of the barrel 12. For example, the proximal seal 54 can include a proximal ridge 58 or O-ring extending from a proximal surface 60 of the proximal seal 54, which is positioned in a corresponding groove 62 of the distal end 16 of the barrel 12 and a distal ridge 64 extending from a distal surface 66 of the proximal seal 54 positioned in a corresponding groove 68 on a proximal end 30 of the cap 24. The proximal ridge 58 or O-ring surrounds the distal opening 18 creating a seal around the opening 18. The seal created by the proximal ridge 58 or O-ring can prevent fluid from collecting under the proximal seal 54, helping to ensure that fluid passing through the distal opening 18 moves to the flow channel 28 of the connector 26.

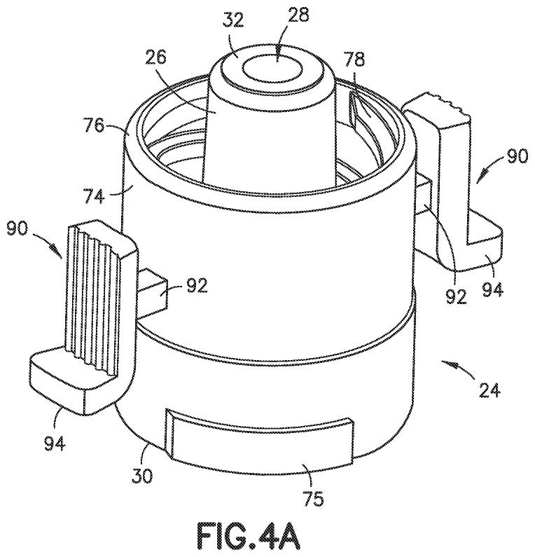

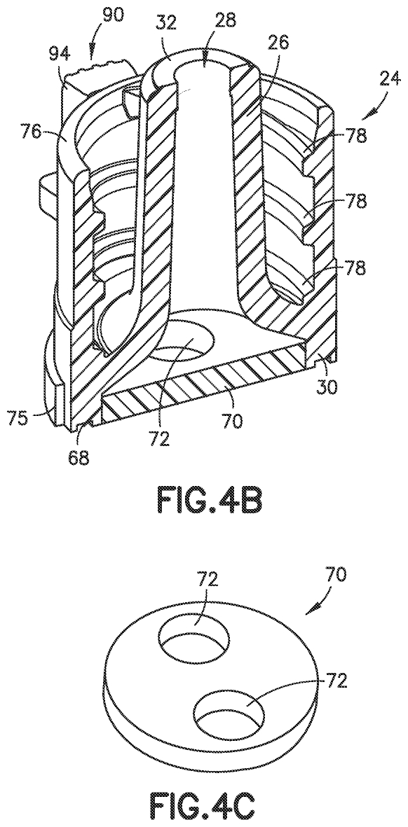

With reference to FIGS. 1A and 1B, as well as FIGS. 4A and 4B, the syringe 10 further comprises the cap 24 connected to the distal end 16 of the barrel 12. For example, as described in further detail herein, the barrel 12 can include locking structures, such as slots, grooves, detects, protrusions, ridges, or similar structures for providing a mechanical engagement between the cap 24 and the barrel 12, thereby securing the cap 24 to the barrel 12 proximate to the distal opening 18 of the barrel 12. The cap 24 comprises the stem or connector 26 defining the flow passage or flow channel 28, which permits fluid flow from the barrel 12 through the connector 26. As previously described, in some examples, the connector 26 of the cap 24 comprises a male luer connector. In other examples, the connector 26 can be a male threaded connector, snap connector, elongated tube, or similar connectors, as are known in the art. As used herein, a “luer connector” refers to a connector that includes a tapered portion (i.e., a luer taper) for creating a friction engagement between the connector 26 and a tapered cavity, such as a cavity of a female luer connector, configured to receive and engage the male connector 26. Accordingly, the connector 26 of the present disclosure can be a tapered elongated structure where an outer diameter of a proximal end of the connector 26 is wider than an outer diameter of a distal tip 32 of the connector 26. The connector 26 can be configured to be connected directly or indirectly to a female connector, which forms a fluid port, valve, or another terminal access portion of a VAD. In particular, a common type of fluid port of a VAD is a pierceable septum or pre-slit septum made of rubber or another elastomeric material. The septum can be pierced or opened by a blunt elongated member or a frusto-conically shaped tip, such as the distal tip 32 of the connector 26, in order to provide fluid communication between the interior of the barrel 12 and the VAD for infusing fluids to or withdrawing fluids from a catheter of the VAD.

In some examples, the cap 24 further comprises a separate seal (referred to herein as a distal seal 70) inserted into a proximal end or proximal opening 30 of the cap 24. Similar to the proximal seal 54, the distal seal 70 comprises a flat sheet or disk having a diameter that matches an inner diameter of the cap 24. The distal seal 70 comprises openings or through holes 72 that can be similar in shape and position as the holes 56 of the proximal seal 54. As shown in FIGS. 5A-5D, the distal seal 70 is positioned immediately distal to the proximal seal 54 and, in some cases, can be in face-to-face contact with the proximal seal 54. As described in further detail herein, the proximal seal 54 is generally a stationary seal 54 and does not move or rotate as the cap 24 moves from the closed position to the open position. By contrast, the distal seal 70, which is inserted in the proximal opening 30 of the cap 24, is configured to rotate along with the cap 24, thereby moving the distal seal 70 from a closed position to the open position.

Both the proximal seal 54 and the distal seal 70 include the holes 56, 72 for permitting fluid flow from the barrel 12 to the connector 26. For example, as shown in FIGS. 3A and 4C, the proximal seal 54 and the distal seal 70 can each include two similarly sized through-holes 56, 72. The through-holes 56, 72 are each offset from the longitudinal axis L1 of the syringe 10 and are not aligned with the distal opening 18 of the barrel 12. Further, when the cap 24 is in the closed position, the holes 56 of the proximal seal 54 are not aligned with the holes 72 of the distal seal 70, as shown in FIGS. 5A and 5B. When the cap 24 is in the open position (shown in FIGS. 5C and 5D), the holes 56 of the proximal seal 54 and the holes 72 of the distal seal 70 are axially aligned so that fluid from the barrel 12 passes through the holes 56, 72 of the proximal seal 54 and the distal seal 70 to the flow channel 28 of the connector 26.

In some examples, the cap 24 further comprises a shield 74 extending about the connector 26. The shield 74 can be an annular or tubular structure having a proximal end connected to a proximal end of the connector 26 and an open distal end 76 that encloses or surrounds the connector 26. The shield 74 comprises an inner surface with threads 78 configured to engage threads of a female connector for securing the syringe 10 and cap 24 to the female connector. For example, the female connector can be a female needless connector, such as a female luer connector, threaded connector, or snap connector, as are known in the art. As previously described, the threads 78 of the shield 74 can be configured to mate to female connector threads with a width at a crest of each thread of from about 0.3 mm to about 1.0 mm and a width at a root of the thread from about 0.5 mm to about 1.2 mm.

The distal tip 32 or end of the connector 26 can extend distally beyond the open distal end 76 of the shield 74, such that the distal end or tip 32 of the connector 26 can be inserted through a cover or septum over an opening or fluid port of the female connector. In some examples, the cap 24 can be configured to rotate in a first direction in order to move the cap 24 from the closed position to the open position. The threads 78 on the inner surface of the shield 74 can be arranged such that the cap 24 and syringe 10 are rotated in an opposite or second direction in order to secure the cap 24 and the syringe 10 to the female connector for expelling fluid form the syringe 10 to the VAD.

With continued reference to FIGS. 1A and 1B, as well as FIGS. 2A-2C, the barrel 12 can further comprise structures for securing the cap 24 over the distal opening 18 of the barrel 12 so that fluid flows through the distal opening 18 to the flow passage or flow channel 28 of the connector 26. For example, the barrel 12 can comprise an extension portion 80 extending distally from the distal end 16 of the barrel 12. As shown in FIGS. 1A and 1B, the cap 24 is connected to and at least partially enclosed by the extension portion 80. The extension portion 80 can be an annular or tubular structure comprising a proximal end 82 extending from the distal end 16 of the barrel 12, an open distal end 84, and an annular sidewall 86 extending between the proximal end 82 and the distal end 84. In some examples, the cap 24 can be inserted or press fit into the extension portion 80 through the open distal end 84 of the extension portion 80. The extension portion 80 can be integrally formed with other portions of the barrel 12 or can be a separate part that is connected to other portions of the barrel 12 by an adhesive, ultrasonic welding, or other suitable connecting processes, as are known in the art.

In some examples, the extension portion 80 comprises one or more slots 88 or openings extending through the sidewall 86 of the extension portion 80. The cap 24 can include one or more tabs 90, which extend radially outwardly from the shield 74 through the slots 88 of the extension portion 80, for rotating the cap 24 relative to the barrel 12. For example, as shown in FIG. 4A, the cap 24 includes two tabs 90. Each tab 90 includes a beam portion 92, which is configured to extend through one of the slots 88 of the extension portion 80, and a grip or handle 94, which can be manipulated by the practitioner to move the cap 24 from the closed position to the open position. In some examples, the handle 94 can include surfaces including ridges, protrusions, and similar structures to make the tabs 88 easier to grasp and manipulate. In particular, the tabs 90 can be configured so that the practitioner can grasp the tab 90 between a thumb and forefinger for rotating the cap 24 between the closed position and the open position. For example, the practitioner may grasp or press the tabs 90 causing the tabs 90 to slide through the slots 88, which causes the cap 24 to rotate from the closed position to the open position so that fluid can flow from the barrel 12 of the syringe 10 through the cap 24.

In some examples, the slots 88 can include an axial portion or segment extending axially through the sidewall 86 from the distal end 84 of the extension portion 80. The axial portion or segment allows the cap 24 to be inserted into the extension portion 80 with the tabs 90 protruding through the slot 88. The slot 88 also includes a circumferential portion or segment for rotating the cap 24 relative to the barrel 12 between the closed position and the open position.

With specific reference to FIG. 2C, the inner surface of the extension portion 80 can include structures for securing the proximal seal 54 in place seated at the proximal end 82 of the extension portion 80. For example, as shown in FIG. 2C, the extension portion 80 can comprise protrusions, such as axially extending ridges 96, ribs, columns, or similar structures, on the inner surface of the extension portion 80 for restricting rotation of a proximal seal 54 positioned over the distal opening 18 of the barrel 12. As show in FIG. 3A, the proximal seal 54 can include cut-outs 55, slots, or grooves positioned to receive the ridges 96 on the inner surface of the extension portion 80. The engagement between the ridges 96 and the cut-outs 55 of the proximal seal 54 maintains positioning of the proximal seal 54 and, in particular, prevents the proximal seal 54 from rotating as the cap 24 rotates between the closed position and the open position.

As shown in FIG. 2C, the extension portion 80 can also include a raised circumferential protrusion, such as a protruding ridge or locking ring 98. The locking ring 98 can be positioned to ensure that the cap 24 and the proximal seal 54 remain seated against the distal end 16 of the barrel 12, preventing the cap 24 and proximal seal 54 from lifting away from the distal opening 18 of the barrel 12 as the cap 24 rotates between the closed position and the open position. For example, the cap 24 can include a proximal protrusion 75 that extends radially outward from the outer surface of the shield 74. When the cap 24 is being inserted into the extension portion 80, the protrusion 75 deflects away from the locking ring 98 so that the cap 24 can be moved through the extension portion 80. When the cap 24 is fully seated in the extension portion 80, the protrusion 75 snaps into place forming a locking engagement with the locking ring 98, which secures the cap 24 in the proximal end 82 of the extension portion 80.

As previously described, the distal end 16 of the barrel 12 can also include the annular groove 62 (shown in FIG. 2C), which receives the ridge 58 or O-ring extending from the proximal surface 60 of the proximal seal 54, which also contributes to maintaining positioning of the proximal seal 54 within the extension portion 80 of the barrel 12. As previously described, the ridge 58 or O-ring also seals the distal opening 18 preventing fluid from collecting between the distal end 16 of the barrel 12 and the proximal seal 54.

Method of Expelling Fluid from a Syringe with an Integrated Cap

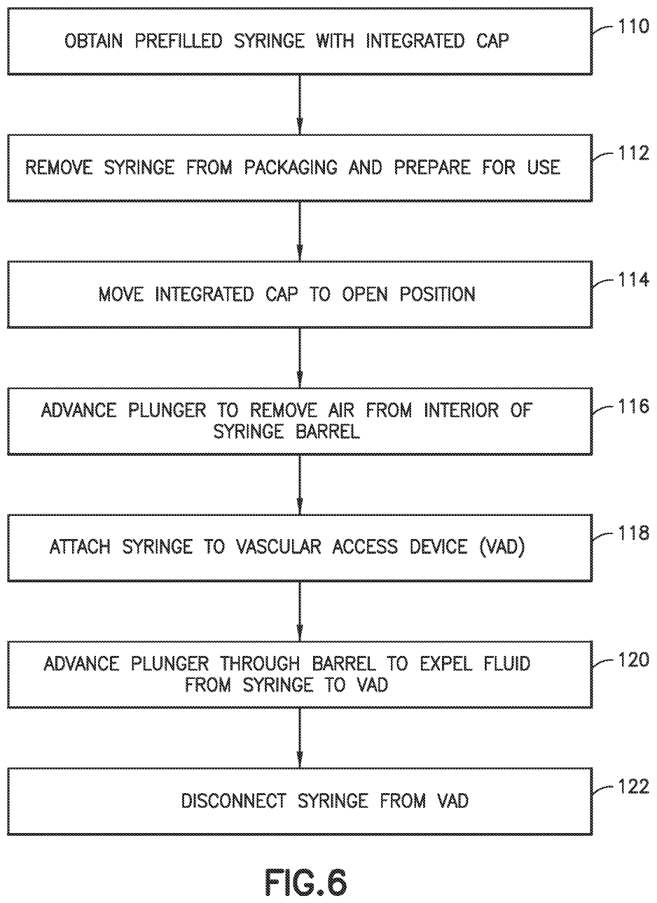

A flow chart illustrating steps for using the syringe 10 to expel fluid to a VAD is shown in FIG. 6. As shown in FIG. 6, at step 110, a practitioner initially obtains a prefilled syringe 10, in which the interior of the syringe barrel 12 is filled with a predetermined volume of the medical fluid F. For example, the prefilled syringe 10 can contain about 1 mL to about 50 mL or, preferably, about 5 mL to 20 mL of the medical fluid F. The cap 24 of the prefilled syringe 10 is initially in the first or closed position to ensure that the medical fluid F remains in the interior of the barrel 12 preventing contamination of the fluid and/or fluid leaks. In this initial or prefilled position, the stopper 38 is positioned proximate to the proximal end 14 of the barrel 12 and the plunger rod 40 extends proximally from the stopper 38 and through the proximal end 14 of the barrel 12.

At step 112, the practitioner prepares the syringe 10 for use by, for example, removing any packaging from the syringe 10 and removing a plunger cap, if present, that holds the plunger rod 40 in place. In the initial position, the cap 24 is in the closed position meaning that the fluid cannot pass through the connector 26. Instead, as shown by the arrows A1 in FIGS. 5A and 5B, the fluid is blocked from passing into the flow channel 28 of the connector 26 by the distal seal 70 because the holes 72 of the distal seal 70 are not aligned with the holes 56 of the proximal seal 54.

At step 114, when ready to perform a flush or injection procedure, the practitioner moves the cap 24 to the open position. For example, the practitioner may grasp one or more of the tabs 90 of the cap 24 between a thumb and forefinger and slide the tabs 90 through the slots 88 of the extension portion 80, as shown A3 (in FIG. 1A), which causes the cap 24 to rotate from the closed position to the open position. Moving the cap 24 to the open position establishes fluid communication between the interior of the syringe barrel 12 and the flow passage or flow channel 28 of the connector 26 so that the medical fluid F can be expelled from the syringe 10.

At step 116, the practitioner next advances the stopper 38 through the barrel 12 by a small amount to remove air from the interior of the syringe barrel 12 by, for example, pressing on the thumb plate 52 of the plunger rod 40 while grasping the finger flange 36 of the barrel 12 between, for example, a ring finger and an index fingers. At step 118, once any air is removed from the interior of the syringe barrel 12, the practitioner next attaches the connector 26 of the cap 24 to the VAD. For example, the practitioner may insert the distal end or distal tip 32 of the connector 26 into a corresponding port or valve of the VAD, in particular into a port or valve of a female needleless connector. Along with moving the syringe 10 distally to press the connector 26 into the septum of the female connector, the practitioner may also rotate the syringe 10 and integrated cap 24 in a direction A4 (shown in FIG. 1A) so that the threads 78 on the inner surface of the shield 74 engage corresponding threads on the female connector to secure the syringe 10 and integrated cap 24 to the female connector. As previously described, the direction of rotation for securing the shield 74 of the cap 24 to the female connector (shown by arrow A4 in FIG. 1A) may be opposite the direction of rotation (shown by arrow A3 in FIG. 1A) to move the cap 24 from the closed position to the open position.

At step 120, with the cap 24 in the open position and attached to the VAD, the practitioner can move the stopper 38 in the distal direction (shown by arrow D in FIG. 1A) through the interior of the syringe barrel 12 using the plunger rod 40 to expel the medical fluid F from the syringe barrel 12 and through the flow passage or flow channel 28 of the connector 26. For example, as previously described, the practitioner may press the thumb press plate 52 of the plunger rod 40 with the thumb while grasping the finger flange 36 of the syringe barrel 12 to move the plunger rod 40 in the distal direction (shown by arrow D in FIG. 1A), which causes the stopper 38 to move distally through the syringe barrel 12 to force the medical fluid F from the syringe barrel 12, as shown by arrows A2 (shown in FIGS. 5C and 5D). The practitioner continues to move the stopper 38 distally through the barrel 12 until all or substantially all of the medical fluid F is expelled from the barrel 12 through the flow passage or flow channel 28 of the connector 26 to the VAD.

At step 122, after the fluid F is expelled from the syringe barrel 12 through the connector 26, the syringe 10 can be disconnected from the VAD by rotating the syringe 10 in the direction A3 (shown in FIG. 1A) to release the threads 78 of the shield 74 from corresponding threads of the female connector.

As previously described, the prefilled syringe 10 of the present disclosure can be filled with a flush solution and used for performing a primary or pre-flush of the patient line of a VAD. In that case, after the flush solution is expelled from the syringe 10, the syringe 10 can be disconnected from the female connector and another syringe containing another medical fluid, such as a therapeutic agent, can be connected to the female connector for providing the therapeutic agent to the patient. In other examples, the prefilled syringe 10 of the present disclosure can contain a dose of the therapeutic agent to be delivered to the patient through the VAD. In that case, the syringe 10 can be attached to the female connector after flushing in order to provide the therapeutic agent to the patient. In other examples, the prefilled syringe 10 of the present disclosure can contain the flush solution and can be used for a post-flush action. For example, the prefilled syringe 10 can be connected to the VAD after the therapeutic agent is delivered to the patient through the VAD. The flush solution can be expelled from the prefilled syringe 10 through the connector 26 and the VAD to flush any remaining therapeutic agent in the VAD to the patient in order to ensure that a full dose of the therapeutic agent is provided to the patient. The post-flush procedure can also ensure that the patient line is clean by removing any debris or other contaminants from the patent line.

While examples of the syringe 10 comprising the integrated cap 24 and methods of the present disclosure are shown in the accompanying figures and described hereinabove in detail, other examples will be apparent to, and readily made by, those skilled in the art without departing from the scope and spirit of the invention. Accordingly, the foregoing description is intended to be illustrative rather than restrictive. The invention described hereinabove is defined by the appended claims and all changes to the invention that fall within the meaning and the range of equivalency of the claims are to be embraced within their scope.

1. A syringe comprising:
a barrel comprising a proximal end, a distal end comprising a distal opening, and a sidewall extending between the proximal end and the distal end;
a plunger positioned in the barrel for expelling fluid from the barrel through the distal opening; and
a cap connected to the distal end of the barrel comprising a connector defining a flow channel through the cap,
wherein the cap is movable relative to the barrel between a first position, where fluid flow through the distal opening of the barrel to the flow channel of the connector is prevented, and a second position where fluid flow through the distal opening of the barrel to the flow channel of the connector can occur.

2. The syringe of claim 1, wherein the cap rotates relative to the barrel about a longitudinal axis of the barrel to move from the first position to the second position.

3. The syringe of claim 1, wherein the cap is configured to be manually moved from the first position to the second position.

4. The syringe of claim 3, wherein the syringe is capable of single-handed operation, both to move the cap from the first position to the second position and to expel fluid from the barrel through the flow channel of the connector by moving the plunger through the barrel.

5. The syringe of claim 1, wherein the plunger comprises a stopper positioned in the barrel and a plunger rod extending proximally from the stopper through the proximal end portion of the barrel.

6. The syringe of claim 1, wherein the connector of the cap comprises at least one of a luer connector, a threaded connector, or a snap connector.

7. The syringe of claim 1, further comprising at least one seal positioned between the distal opening of the barrel and the cap, the at least one seal comprising at least one hole for fluid flow from the barrel to the flow channel of the connector.

8. The syringe of claim 7, wherein the at least one seal comprises at least two holes, each of which are offset from a longitudinal axis of the syringe.

9. The syringe of claim 7, wherein the at least one seal comprises a disk formed from a thermoplastic elastomer comprising at least two holes, which are spaced apart from a longitudinal axis of the barrel and are not aligned with the distal opening of the barrel.

10. The syringe of claim 9, wherein the disk comprises a proximal ridge extending from a proximal surface of the disk positioned in a corresponding groove of the distal end of the barrel and a distal ridge extending from a distal surface of the disk positioned in a corresponding groove on a proximal end of the cap.

11. The syringe of claim 7, wherein the at least one seal comprises a proximal stationary seal positioned between the distal end of the barrel and a proximal end of the cap, and a distal rotatable seal positioned within a proximal open end of the cap.

12. The syringe of claim 11, wherein the proximal seal and the distal seal each comprise at least one hole that is offset from a longitudinal axis of the syringe and is not aligned with the distal opening of the barrel, and
wherein, when the cap is in the second position, the at least one hole of the distal seal aligns with the at least one hole of the proximal seal so that fluid from the barrel passes through the holes of the proximal seal and the distal seal to the flow channel of the connector.

13. The system of claim 1, wherein the cap further comprises a shield extending about the connector, the shield comprising an inner surface comprising threads configured to engage threads of a female connector for securing the syringe and cap to the female connector.

14. The syringe of claim 13, wherein the barrel further comprises an extension portion extending distally from the distal end of the barrel, and wherein the cap is connected to and at least partially enclosed by the extension portion.

15. The syringe of claim 14, wherein the extension portion comprises a proximal end extending from the distal end of the barrel, an open distal end, and an annular sidewall extending between the proximal end and the distal end, and
wherein the cap comprises a locking ring configured to engage the cap so that the cap remains seated at the proximal end of the extension portion.

16. The syringe of claim 14, wherein the cap comprises at least one tab extending radially outward from the shield for rotating the cap relative to the barrel,
wherein the extension portion comprises at least one slot, and
wherein the at least one tab of the cap extends through the slot so that the at least one tab can be grasped by a user to rotate the cap relative to the barrel.

17. The syringe of claim 14, wherein the extension portion further comprises protrusions on an inner surface of the extension portion for restricting rotation of a seal positioned over the distal opening of the barrel, and
wherein the protrusions comprise axially extending ridges, ribs, or columns on the inner surface of the extension portion.

18. The syringe of claim 1, wherein a direction of rotation for connecting the syringe to a female connector is opposite a direction of rotation to move the cap from the first position to the second position.

19. A prefilled flushing syringe, comprising:
a syringe having a barrel comprising a proximal end, a distal end comprising a distal opening, and a sidewall extending between the proximal end and the distal end;
a plunger positioned in the barrel for expelling fluid from the barrel through the distal opening; and
a cap connected to the distal end of the barrel comprising a connector defining a flow channel through the cap,
wherein the cap is movable relative to the barrel between a first position, where fluid flow through the distal opening of the barrel to the flow channel of the connector is prevented, and a second position where fluid flow through the distal opening of the barrel to the flow channel of the connector can occur;
and
a predetermined volume of a fluid disposed in the barrel of the pre-filled syringe,
wherein the cap is initially in the first position, thereby preventing the fluid in the barrel from passing through the open distal end portion of the barrel to the flow channel of the connector.

20. A method for expulsion of fluid from a syringe, comprising:
providing a syringe having a barrel comprising a proximal end, a distal end comprising a distal opening, and a sidewall extending between the proximal end and the distal end, a plunger positioned in the barrel for expelling fluid from the barrel through the distal opening, and a cap connected to the distal end of the barrel comprising a connector defining a flow channel through the cap, wherein the cap is movable relative to the barrel between a first position, where fluid flow through the distal opening of the barrel to the flow channel of the connector is prevented, and a second position where fluid flow through the distal opening of the barrel to the flow channel of the connector can occur;
moving the cap from the first position to the second position, such that fluid flow from the barrel to the flow channel of the connector is permitted;
attaching the connector of the cap to a vascular access device; and
moving the plunger through the barrel to expel the fluid from the barrel and through the flow channel of the connector to the vascular access device.
